(19) United States
(12) Patent Application Publication
(10) Pub. No.: US 2025/0135242 A1
(43) Pub. Date: May 1, 2025

(54) MONITORING SYSTEM WITH A CATALYTIC GAS MEASURING DEVICE FOR PILOTS

(71) Applicant: Dräger Safety AG & Co. KGaA, Lübeck (DE)

(72) Inventors: Tilman von BLUMENTHAL, Lübeck (DE); Hans-Ullrich HANSMANN, Lübeck (DE); Henning GERDER, Lübeck (DE); Robert JAHNS, Lübeck (DE); Christoph OSTERLOH, Lübeck (DE)

(73) Assignee: Dräger Safety AG & Co. KGaA, Lubeck (DE)

(21) Appl. No.: 18/925,671

(22) Filed: Oct. 24, 2024

(30) Foreign Application Priority Data
Oct. 25, 2023 (DE) 10 2023 129 372.9

(51) Int. Cl. A62B 7/14; A62B 9/00; G01N 33/00

(52) U.S. Cl. CPC A62B 7/14; A62B 9/006; G01N 33/0011; G01N 33/0063

(57) ABSTRACT

A monitoring system (100) with an associated catalytic gas measuring device (72) is used in a breathing gas supply system in aircraft. During a coordinated operation of the monitoring system (100) and the catalytic gas collection device (72), quantities of breathing gas mixture (10) are supplied to the catalytic gas measuring device (72). The catalytic gas measuring device (72) is configured for metrological detection of a selected target gas with the aid of conversion factors (79).

### CROSS REFERENCE TO RELATED APPLICATIONS

This application claims the benefit of priority under 35 U.S.C. § 119 of German Application 10 2023 129 372.9, filed Oct. 25, 2023, the entire contents of which are incorporated herein by reference.

### TECHNICAL FIELD

The present invention pertains to a monitoring system with a catalytic gas measuring device for pilots or passengers of aircraft devices. Aircraft or aircraft devices are to be understood as aircraft or helicopters in civil or military aviation, such as passenger aircraft in scheduled or chartered flights, as well as ultrafast aircraft close to or above the supersonic speed range. In particular, flights with jet aircraft at supersonic speeds and/or at altitudes above 15,000 meters above the seabed (sea level) place great demands on the fitness to fly of flight personnel. Fitness to fly with physical and mental fitness, alertness, concentration and vigilance must be ensured at all times at high altitudes, rapid flight maneuvers or flight attitudes, such as turns, dives, inverted flight at high speeds and high accelerations. In addition to the personal and health conditions of the pilot and reliable equipment of the aircraft, a reliable supply of clean and safe breathing air for the pilot is also very important to ensure safe flight operations.

Breathing air can be supplied by suction from the ambient air—often in the area of the engines or turbines—and/or with a so-called OBOGS system. The supply gas provided in this way is used to supply breathing gas to crews and passengers during a flight with an aircraft. The OBOGS system can be configured as an oxygen enrichment system using an arrangement of molecular sieves based on the principle of pressure swing absorption or as a system with air or pressurized oxygen gas cylinders. As an emergency supply system in the event of a failure of the O₂ main supply, a system for chemical oxygen generation is often also carried on board aircraft.

The breathing gas mixture may also be contaminated with other gas components.

### BACKGROUND

U.S. Pat. No. 11,033,762 B2 discloses a device and a method for monitoring inhaled gases from an aircraft pilot's gas supply using an OBOGS system and a face mask.

U.S. Pat. No. 2,021,405 008 A1 shows a monitoring system for pilots of an aircraft. The monitoring system makes it possible to monitor the quantities of gas supplied to the pilot with regard to the oxygen or carbon dioxide concentration. For this purpose, a gas mixture from the pilot's face mask is fed to an analyzer by means of a suction measurement.

A measuring system with a preliminary measurement as a “pre-check” with a subsequent specific measurement is described in EP 2 788 751 B1. There, changes in the gas composition in a gas mixture are determined by means of an ultrasonic measurement based on changes in the speed of sound in a gas mixture. Based on this, a measurement with an optical gas sensor is then activated in order to further analyze the gas mixture.

U.S. Pat. No. 11,401,945 shows a catalytic gas sensor. Catalytic gas sensors in the form of heat tone sensors are suitable for detecting the presence of explosive gases or explosive gas mixtures in a measuring environment. The essential component of a heat tone sensor is formed by a small measuring element—often referred to as a pellistor—usually an electrically heated platinum coil, which is coated with a ceramic material with an outer catalytic coating of a precious metal, for example rhodium.

When a combustible gas/air mixture flows over the hot catalytic converter surface, combustion occurs and the resulting heat increases the temperature and therefore the electrical resistance of the measuring element. The change in resistance can be directly related to the gas concentration in the surrounding atmosphere. Catalytic gas sensors are also suitable for detecting rapid changes in gas concentrations; response times (T₁₀₋₉₀) for catalytic gas sensors are in the range of 10 and 30 seconds.

The present invention relates generally to a gas measuring device for an extended gas analysis of the breathing gas supply of pilots with an on-site/pre-use calibration for a monitoring system, as shown, for example, in U.S. Pat. No. 2,021,405 008 A1.

Such a monitoring system enables the monitoring of gas quantities of the breathing gas supply of aircraft pilots with regard to the oxygen concentration in the breathing gas during flight operations. For this purpose, a gas mixture from the pilot's face mask and/or from the cockpit is fed to an oxygen sensor in the monitoring system by means of a suction measurement. This oxygen sensor is configured as a paramagnetic oxygen sensor. The paramagnetic oxygen sensor has thermoelectric measuring elements and is thus able to determine the oxygen concentration in the breathing gas by utilizing the paramagnetic effect of the thermal conduction of oxygen on the basis of changes in thermal conductivity under the influence of magnetic field modulation. The utilization of the paramagnetic effect makes the determination of the oxygen concentration in the total mixture of the breathing gas largely independent of the presence of other gases.

From the German patent application DE 10 2023 121 409.8, which has not yet been published (or disclosed), a variant of a monitoring system for monitoring a pilot and/or co-pilot is described and shown, which is configured to determine a condition as to whether there is a presence of foreign gas components in addition to proportions of oxygen, carbon dioxide or nitrogen in a breathing gas mixture. This monitoring system according to DE 10 2023 121 409.8 can determine on the basis of the thermal conductivity information whether a situation exists in which certain quantities of impurities with other gases or gas mixtures are present in the breathing gas mixture. The monitoring system cannot directly determine the nature of the impurities in the breathing gas mixture, but it is possible to identify a particular situation with suspected impurities. Using the paramagnetic measuring device and evaluating thermoelectric voltages, this monitoring system enables an initial analysis of a situation to determine whether additional components such as soot, oil, oil vapors or other gas components are present in the breathing gas mixture in a certain concentration, which are to be regarded as undesirable foreign gases.

For a qualitatively precise determination of the type of impurities in the breathing gas, additional and precise measuring systems, such as a gas chromatograph, must be installed in the aircraft in addition to the monitoring system. However, gas chromatographs cannot be used in the aircraft simply because of the limited space available and the operating requirements.

In addition, certain measuring systems with sensors whose functionality is significantly impaired by vibrations, acceleration, extreme altitude differences and rapid pressure changes during flight cannot be used in an aircraft environment. These include, for example, sensors with moving parts, such as chopper wheels, as well as electrochemical sensors or chemical analysis tubes.

### SUMMARY

It is an object of the invention to provide a monitoring system with an additional gas measuring device for an extended analysis.

A further task is to realize a combination of the monitoring system with the additional gas measuring device and to specify a coordinated operation of the monitoring system and gas measuring device.

This problem is solved by a system comprising a monitoring system configured to monitor a gas composition of breathing gases or breathing gas mixtures in aircraft and a catalytic gas measuring device.

The problem is solved in particular by a monitoring system for extended gas analysis with a paramagnetic measuring device and a catalytic gas measuring device with at least one catalytic gas sensor.

The present invention supplements the possibilities of the monitoring system shown in U.S. Pat. No. 2,021,405 008 A1 with an additional, specially configured gas measuring device in order to realize an extended gas analysis in this way (US20210405008 A1 is incorporated herein by reference).

According to the invention, the extended gas analysis by the gas measuring device is carried out using a catalytic gas sensor system.

To solve the aforementioned problem, the invention proposes a monitoring system for monitoring the gas composition of breathing gases in aircraft or aircraft devices in combination with a catalytic gas measuring device. The catalytic gas measuring device is characterized by the fact that it—as well as catalytic gas sensors arranged in the catalytic gas measuring device—can be calibrated in a special way before the start of flight operation.

Catalytic gas sensors are particularly suitable for the metrological detection of explosive gases, gas mixtures, fluids or vapors and the associated evaluation of situations in flight operations with regard to potentially hazardous situations, explosion hazards and the associated risks to the health and life of the pilot.

The combination of monitoring system and catalytic gas measuring device according to the invention enables the catalytic gas measuring device to be checked by a control unit of the monitoring system in situations in which foreign gas components may be present in a breathing gas or breathing gas mixture.

Catalytic gas sensors or catalytic gas measuring devices can be used to detect different gases by means of special calibrations. In most cases, the focus is on monitoring explosive gases or gas mixtures.

The catalytic gas measuring device can also be prepared for fluids, vapors, gases or gas mixtures that can be expected at the measuring or operating site in the further course of time by means of so-called on-site calibrations directly at the measuring or operating site. During on-site calibrations, calibration gases are supplied to the catalytic gas sensor in a defined quantity and with a known concentration from a small container in a calibration procedure using suitable mobile calibration devices, an output signal of the catalytic gas sensor is recorded and analyzed by measurement and a specific conversion factor is determined by calculation on the basis of the output signal and the known concentration of the calibration gas, which is representative and characteristic of the calibration gas.

Here is an example: In a production plant, substances containing solvents are often used as adhesives, resins or lacquers in certain processing steps for painting or bonding components.

Data sheets for the substances show, for example, that toluene (C₆H₅CH₃) is also added as a solvent. Toluene forms highly flammable vapor-air mixtures, toluene is also harmful to health, inhalation of toluene vapors can lead to symptoms such as fatigue, malaise, sensory disturbances, impaired coordination of movement and loss of consciousness.

To monitor concentrations of toluene, gas mixtures containing toluene or toluene vapors in the production plant, several measuring systems with catalytic gas sensors are positioned in the production plant. By carrying out on-site calibrations, specific monitoring of the concentration of toluene in the ambient air can be carried out. For on-site calibration, a small amount of liquid with a known concentration of toluene is nebulized in a defined air flow using a mobile calibration device and fed to the catalytic gas sensor for a defined period of time, then a specific conversion factor that is representative and characteristic for the metrological detection of toluene with this catalytic gas sensor is determined by calculation from the output signal of the catalytic gas sensor and the known concentration of the calibration gas. This specific conversion factor is characteristic and representative for the metrological detection of toluene in the now calibrated measuring system. The specific conversion factor can be stored in the measuring system, and in subsequent measuring operation the measuring system can then provide, document and/or output on a display unit a current concentration value for a concentration of toluene in the gas mixture, breathing gas or breathing gas mixture on the basis of the output signal and the representative and characteristic specific conversion factor. In addition, upper threshold values of alarm limits can also be set, for example, and alarms can be triggered by the measuring system, either acoustically or visually, if these are exceeded. Examples of threshold values are the lower explosion limit (LEL) with 1.1% vol. %, the upper explosion limit (UEL) with 7.8% vol. %, and threshold values can also be defined in relation to possible health hazards.

In addition to its use as a solvent, toluene is also used in the manufacture of explosives (TNT=trinitrotoluene) and, alongside benzene, toluene is also a component of fuels such as gasoline (petrol). This makes it clear that monitoring toluene, for example, as well as other substances used in the operation of an aircraft as components of fuels, operating materials, lubricants or coolants, can be useful both for reasons of explosiveness and because of possible health risks for the pilot.

The application methodology described above—and using the example—means that catalytic gas sensors can be used by means of calibrations to determine a variety of gases or substances—especially explosive substances—in a gas sample by measurement.

The present invention takes up this possibility of determining the concentration by means of on-site calibrations in order to enable the metrological determination of selected other gases in the breathing gas or breathing gas mixture in a monitoring system for aircraft pilots. These on-site calibrations can also be referred to as adjustments.

The selected fluids, gases, gas mixtures as well as selected vapors can be and are referred to as target gases or target gas in the context of the present invention.

According to the invention, a combination of a monitoring system with a paramagnetic oxygen sensor and a catalytic gas measuring device with at least one catalytic gas sensor is suitably configured. The combination of monitoring system and catalytic gas measuring device according to the invention is configured for this purpose as described in more detail below.

The monitoring system has a paramagnetic oxygen sensor and a control unit.

The control unit is configured to organize, monitor, control or regulate a process of metrological monitoring of the gas composition of air, breathing air or breathing gases in aircraft or aircraft devices.

A catalytic gas measuring device with at least one catalytic gas sensor and an evaluation unit is assigned to the monitoring system.

The control unit is also configured to coordinate operation of monitoring systems and the catalytic gas measuring device. For this purpose, the control unit can activate and/or deactivate the catalytic gas measuring device or components of the catalytic gas measuring device.

The combination of monitoring system and catalytic gas measuring device has at least one module for gas transport. The at least one module for gas transport, by means of a sample gas line for supplying quantities of breathing gas or breathing gas mixture, sucks in a gas sample from a measuring location, in particular at the breathing mask of the pilot and/or from the cabin or cockpit. The gas sample is conveyed to the monitoring system.

For this purpose, the catalytic gas measuring device gas transport module is pneumatically and/or fluidically connected to the measuring point for gas transport using the sample gas line. The gas sample can reach the paramagnetic oxygen sensor through a gas inlet on the monitoring system. In addition, the gas sample is fed to the catalytic gas measuring device and the catalytic gas sensor and made available for analysis.

The at least one gas transport module can be configured to supply quantities of breathing gas or breathing gas mixture from a measuring location to the catalytic gas measuring device with the at least one catalytic gas sensor by means of the same sample gas line or by means of a further sample gas line.

The at least one gas transport module or a further gas transport module can be configured for an inflow of defined quantities of a test gas to the catalytic gas measuring device with the at least one catalytic gas sensor by means of the same sample gas line, the further sample gas line or a calibration gas line.

With the paramagnetic oxygen sensor and the control unit, the monitoring system is configured to perform qualitative and quantitative measurement of the concentration of oxygen in the supplied quantities of breathing gas or breathing gas mixture.

The catalytic gas measuring device is configured with the at least one catalytic gas sensor and the evaluation unit for metrological determination of at least one further fluid, gas or gas mixture selected as the target gas in the supplied quantities of breathing gas or breathing gas mixture.

The evaluation unit is configured to determine a target gas identifier which indicates the presence of the target gas in the breathing gas or breathing gas mixture and is also configured to provide the target gas identifier as an output signal.

For this purpose, an output signal from the catalytic gas sensor is evaluated by the evaluation unit.

According to the invention, at least one calibration gas interface is arranged in or on the monitoring system or the catalytic gas measuring device.

The calibration gas interface is configured to supply predetermined quantities of calibration gas to the catalytic gas sensor.

The calibration interface can be configured in different ways, for example in the form of a slide-in opening or in the form of a drawer.

In preferred embodiments, the calibration interface can be configured as a connection, an insertion or a receptacle for a calibration medium, for example in the form of a cassette. The calibration medium can have a flow chamber or an evaporation chamber with a defined volume. The calibration medium can be configured for coupling or in-coupling a container filled with a calibration gas. In an optional embodiment, the calibration medium can also have means for supplying quantities of a calibration gas into the flow chamber or evaporation chamber.

In a preferred embodiment, the evaluation unit can also be configured to carry out the metrological determination of the presence of the other fluid, gas or gas mixture selected as the target gas in the quantities of breathing gas or breathing gas mixture supplied, taking into account a specific conversion factor.

The specific conversion factor is specifically assigned to the at least one other gas, fluid, gas mixture or group of gases, fluids or gas mixtures selected as a target gas.

The specific conversion factor is representative, specific and/or typical for the at least one target gas.

In this preferred embodiment, a data memory is connected to the evaluation unit. At least one specific conversion factor for the at least one calibration gas associated with the selected target gas is stored in the data memory. The specific conversion factor is used by the evaluation unit to perform a scaling corresponding to the selected target gas on the output signal of the catalytic gas sensor, which is given when the catalytic gas sensor comes into contact with the selected target gas, and to include the conversion factor scaling in the target gas identifier.

The target gas identifier can comprise a concentration, a volume concentration, a volume proportion, a volume ratio, a substance quantity concentration (molarity), a weight proportion or a partial pressure of the target gas in the breathing gas or breathing gas mixture. Further conversions of the concentration, for example in volume, into pressure ratios can be integrated into the specific conversion factor and thus also be included in the determination of the target gas identifier.

In this way, the specific conversion factor can advantageously enable the evaluation unit to make various conversions to formations of the target gas identifier during operation of the catalytic gas measuring device.

Embodiments can also show how the inclusion of the specific conversion factor can be implemented in practice through interaction of the monitoring system with the control unit and the catalytic gas measuring device with the evaluation unit. For example, the evaluation unit can be configured to provide the output signal of the catalytic gas sensor, the target gas identifier or the output signal to the monitoring system, in particular to the calculation and control unit.

Embodiments can show how calibration medium can be configured in interaction with the calibration interface or as an element of the calibration interface.

Calibration medium can be configured as calibration containers for storing a deposit or a quantity of a calibration fluid in fluid or gaseous form or as evaporation trays for evaporating the calibration fluid.

Suitable designs of the container for storing the calibration gas can be configured as a tank, a cartridge, a cylinder or a pressurized gas cylinder. Calibration containers can be configured as calibration gas cylinders or calibration gas bottles in order to store a depot of calibration gas at a pressure level above the ambient pressure, comparable to configurations of a tank of a liquid gas lighter.

In a preferred embodiment, an evaporation chamber is arranged in or on the calibration interface as a calibration medium. The calibration interface is configured to accommodate a calibration gas cylinder. The calibration gas cylinder is configured to store quantities of a calibration gas at a pressure level above the ambient pressure.

In a preferred embodiment, a receptacle for an evaporation chamber is arranged in or on the calibration interface as calibration medium. A container filled with calibration gas can be configured as an evaporation tray in order to store a deposit of calibration gas as a fluid in liquid or gaseous form. The evaporation tray can be configured to hold a deposit of calibration fluid at ambient pressure. The evaporation chamber or the calibration interface can be configured to accommodate an evaporation tray (evaporation dish).

The evaporation tray is configured to store quantities of a calibration fluid. A further gas transport module is arranged in or at the calibration interface or in or at the evaporation chamber, which is configured to transport quantities of calibration gas from the evaporation chamber to the catalytic gas sensor.

In alternative embodiments, the calibration interface can also be configured to be connected by means of a calibration gas line in order to implement a connection between the catalytic gas measuring device and the container with a supply of calibration gas and to enable the provision of calibration gas to the catalytic gas measuring device. At the other end, the calibration gas line is connected directly or indirectly, for example in a serial arrangement to a switching device, valves or conveying means, to a container in which a deposit of a quantity of calibration gas is stored. The calibration interface can be configured as a pneumatic interface, for example to form a pneumatic plug element and a pneumatic bushing as a detachable pneumatic connecting element.

Further embodiments can show how the catalytic gas measuring device can be connected to the monitoring system. For example, the catalytic gas measuring device can be integrated as a module in the monitoring system and can also be arranged as a further, additional module at the gas outlet of the monitoring system.

Embodiments can show how an initiation and/or control of the supply of the gas sample to the catalytic gas measuring device can be configured. Such embodiments relate, for example, to the switching or switching valves and/or changeover valves as well as activations or deactivations of the module for gas transport as well as of further gas conveying means arranged in the gas measuring device, for example pumps, in order to convey the quantities of breathing gas or breathing gas mixture as a gas sample from the monitoring system to the catalytic gas measuring device by means of the sample gas lines as well as quantities of calibration gas by means of the calibration gas lines and thus to provide the catalytic gas sensor for metrological detection according to the heat tone measuring principle.

The supply of the gas sample of breathing gas as well as the supply of quantities of calibration gas to the catalytic gas measuring device can be controlled by the control unit, for example by means of an actively switched valve. In alternative embodiments, gas samples from the face mask and/or the cockpit can be supplied in parallel to both the monitoring system with the paramagnetic measuring system and the catalytic gas measuring device at the same time.

The use of calibrations with calibration gases in the catalytic gas measuring device simplifies the search for and identification of specific gases, vapors or gas mixtures in flight operations for the user in preparation for flight operations by allowing the user to carry out an on-site calibration using a calibration gas suitably selected with regard to the target gas being searched for prior to flight operations.

The application of on-site calibrations is described below in a simplified sequence of steps:
First, a target gas is selected by a user, for example by maintenance personnel at an air base, which may be present as a component in the breathing gas or breathing gas mixture during flight operations.
The calibration gas best suited to the target gas is then selected, for which a suitable specific conversion factor is available for scaling the measured values of the catalytic gas sensor with regard to the target gas.
The gas measuring system is then calibrated on site with the selected calibration gas.
The specific conversion factor for the selected pairing of target gas, calibration gas and the monitoring system and/or the catalytic gas measuring device is then calculated or taken from tables and provided to the catalytic gas measuring device.

To provide the conversion factor, a data memory (data storage) can be arranged on or in the evaluation unit or assigned to the evaluation unit. At least one specific conversion factor associated with the at least one further gas, gas mixture or group of gases or gas mixtures selected as the target gas is stored as a data record (such as data sets) in the data memory.

This means that the catalytic gas measuring device is suitably equipped to qualitatively and quantitatively measure the target gas sought during the subsequent flight operation, determine the target gas identifier and provide the target gas identifier as an output signal.

The catalytic gas sensors can be calibrated in an advantageous way to known auxiliary and operating fluids by knowing the auxiliary and operating fluids used in the aircraft or aircraft devices in different configurations.

This means that the user can calibrate the catalytic gas sensors for the upcoming use directly on site to an expected target gas by means of an on-site calibration without having to know or disclose specific information on auxiliary or operating materials for the design, development and provision of the monitoring system to the manufacturer of the monitoring system. Examples of auxiliary or operating materials include fuels, coolants, lubricants, coatings or paints.

The operator of the aircraft or aircraft device can thus carry out calibrations on catalytic gas sensors for substances before use without having to provide a manufacturer of the monitoring system, the gas measuring device or the catalytic gas sensors with information about the type of substances in question. This type of on-site calibration enables the operator to comply with any confidentiality requirements vis-à-vis third parties with regard to specific expected target gases if, for example, the auxiliary materials or fuels for aircraft are a possible source of the target gases and these auxiliary materials or fuels are based on special and possibly not freely available formulations.

The at least one catalytic gas sensor is preferably closely coupled to the monitoring system by means of a data connection, for example in the form of a bus system, and preferably also connected to its power supply. Since the monitoring system is preferably configured to be mobile and autonomous from the aircraft and is arranged on the clothing of the pilot, e.g. in a vest or jacket pocket, it is essential to use the amount of energy carried in the monitoring system as sparingly as possible.

It makes sense that only those components of the monitoring system and/or the catalytic gas measuring device that are absolutely necessary for the respective task are continuously in operation and/or electrically supplied. Due to the measuring principle with the heated measuring element, catalytic gas sensors cannot necessarily be implemented as low-power sensors. To ensure careful use of the stored electrical energy, which is only available for a limited time during the period of use, it is therefore advantageous not to continuously supply a gas sample to the other sensors, nor to carry out measurement operation, data acquisition or data storage, but only to do so when an exceptional situation occurs that is determined by the monitoring system or based on a special situation, such as an activity of the aircraft crew. If an unusual or special situation occurs, the control unit in the monitoring system can then activate a valve arrangement and/or pump arrangement in the catalytic gas measuring device in such a way that gas quantities are conveyed to the additional sensor system with the catalytic gas sensor by means of the sample gas lines.

At the same time, the additional sensor system is then activated and the gas sample is then analyzed in relation to the unusual or special situation. When the sensor system with the catalytic gas sensor is activated, the control unit in the monitoring system can also switch on the power supply for the other sensors and/or the catalytic gas sensor as required.

In a preferred embodiment, the control unit is configured to carry out a determination as a function of the target gas detection provided by the catalytic gas measuring device as to whether there is a current situation that poses a health risk to an aircraft pilot during operation of the breathing gas supply of the aircraft. If a situation that poses a health risk to the pilot is currently present, the control unit is configured to activate a vibration alarm detector arranged in or on the monitoring system or a vibration alarm detector assigned to the monitoring system.

The vibration detector can be integrated into a housing of a monitoring system configured as a mobile unit or a mobile gas measuring device. In an alternative embodiment, the vibration detector can also be configured as a component in a wristwatch or another mobile device arranged on the wrist of a pilot or co-pilot. In addition to the vibration detector, the wristwatch or the other mobile device arranged on the wrist of a pilot or co-pilot can also have elements for a visual or acoustic alarm of the health-threatening situation and thus be configured to provide an acoustic and/or visual and/or tactile alarm.

In further preferred embodiments, components of the monitoring system and catalytic gas measuring device can be connected to each other by means of an interface arrangement including energy (electrical power) and/or data interfaces and line connections. For example, the control unit can also be configured to coordinate the operation of the catalytic gas measuring device and the monitoring system by means of the energy and/or data interfaces and the line connections.

If, for example, a condition has been detected by the control unit in the monitoring system in interaction with the paramagnetic oxygen sensor by a special evaluation of thermal conductivity situations—as described, for example, in DE 10 2023 121 409.8 (corresponding U.S. application Ser. No. 18/798,948 is incorporated herein by reference)—that a gas, fluid or gas mixture other than oxygen, nitrogen, moisture or water vapor and carbon dioxide may be present in the breathing gas or breathing gas mixture, the control unit can, on the one hand, activate the gas measuring device, i.e. put it into measuring mode, and/or activate switching or changeover device in the monitoring system and/or catalytic gas measuring device in such a way that a supply of a gas sample of breathing gas to the catalytic gas sensor is possible, on the one hand, the control unit can activate the gas measuring device, i.e. put it into measuring mode, and/or activate a switching device (switching means) or changeover device (changeover means) in the monitoring system and/or catalytic gas measuring device in such a way that a gas sample of breathing gas can be supplied to the catalytic gas sensor.

In particularly preferred embodiments, the control unit can be configured to carry out at least one action of the following actions when performing the coordination of the monitoring system and catalytic gas measuring device:
Activation of the switching device or changeover device to start the supply of quantities of breathing gas to the catalytic gas measuring device;
Deactivation of the switching device or changeover device to terminate the supply of quantities of breathing gas to the catalytic gas measuring device;
Activation of components of the catalytic gas measuring system such as evaluation unit, catalytic gas sensor, components for power supply;
Deactivation of components of the catalytic gas measuring system such as the evaluation unit, catalytic gas sensor and components for power supply;
Connection of the electrical power supply to the monitoring system and/or gas detection system or individual components of the monitoring system and/or catalytic gas detection system;
Switching off the electrical power supply to the monitoring system and/or gas detection system or individual components of the monitoring system and/or catalytic gas detection system.

If the additional sensor system is configured as an independent module with its own power supply, the control unit in the monitoring system can activate the catalytic gas measuring device from a standby or idle mode with low energy requirements to a measuring mode in which the catalytic gas sensor is also supplied with electrical energy. Through such measures of energy management and energy control by the control unit in the monitoring system, the energy storage devices of the monitoring system and/or the other sensors required for flight operation can be kept as small as possible in terms of weight, volume and size. This is particularly advantageous for mobile equipment worn on the body or close to the body, for example in terms of wearing comfort and space requirements on clothing and with regard to minimizing restrictions on the pilot's mobility at their workplace. In the event of an unplanned exit from the cockpit using a parachute, it is also advantageous to largely avoid any unnecessary weight on the pilot's clothing as well as restrictions on the pilot's mobility on the parachute.

In a further preferred embodiment, one or more data interfaces can be provided on the monitoring system and/or on the catalytic gas measuring device, which can enable initiation or control of the on-site calibrations by the monitoring system or other systems. It may also be possible to select a calibration gas.

In further preferred embodiments, these data interfaces may be provided to enable the provision of specific information from the monitoring system or other systems to the catalytic gas measurement device.

The special information can include, for example, information on the target gas, the calibration gas, the specific conversion factor, the calibration gas container, alarm limit values, toxicity or explosiveness threshold values.

In a further preferred embodiment, an input unit can be provided on the monitoring system and/or on the catalytic gas measuring device, which allows a selected gas or gas mixture to be entered or selected as the target gas and/or a calibration gas and/or the specific conversion factor.

The input unit can also enable operation, initiation or control of the on-site calibrations. The user can also be able to enter or select a calibration gas or select a calibration gas container. The input unit can also be configured as an interface for wireless data exchange, for example using NFC (Near Field Communication), Bluetooth or RFID technology.

By providing the special information via data interfaces or selecting a target gas at the input unit, the catalytic gas measuring device is prepared for a measuring operation in which qualitative and/or quantitative measurement of the target gas is possible.

In further preferred embodiments, conversion factors for different fluids, gases or gas mixtures can be stored as target gases in connection with suitable calibration gases or calibration fluids in data records in the data memory. The data records with the conversion factors can be stored in tables, data sets or database systems in a sortable manner. The data memory can also be configured as an external data memory, for example as a USB stick, so that during the on-site calibration of the catalytic gas measuring device, information is imported from the external data memory into the evaluation unit of the catalytic gas measuring device in a manner adapted to the subsequent use and the data memory itself is no longer required in subsequent operation.

This type of on-site calibration configuration enables a robust design of the operation of the catalytic gas measuring device with regard to possible confidentiality requirements with regard to the potentially expected target gases, as well as the calibration gases or calibration fluids.

In a further preferred embodiment, an output unit can be arranged in or on the monitoring system and/or in or on the catalytic gas measuring device, which is configured to provide and/or visually output in relation to the selected target gas and/or to the selected calibration gas and/or to output status information in relation to the catalytic gas measuring device.

This allows measured values or gas concentration values of the target gas as well as status information during operation of the catalytic gas measuring device, such as standby or idle mode, active measuring mode, current energy consumption, alarm statuses as well as a charge status of the energy stores to be output to the user as visual messages, for example alphanumerically, in text form or graphical form (GUI).

The invention is explained in more detail in the following description with partial reference to Figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a monitoring system with features forming a part of a system with the monitoring system and catalytic gas measuring device according to the invention;

FIG. 2 is a schematic view of a catalytic gas measuring device with connections to the monitoring system;

FIG. 3 is a schematic view of an alternative catalytic gas measuring device with connections to the monitoring system; and

FIG. 4 is a schematic view of a further alternative catalytic gas measuring device with connections to the monitoring system.

### DESCRIPTION OF PREFERRED EMBODIMENTS

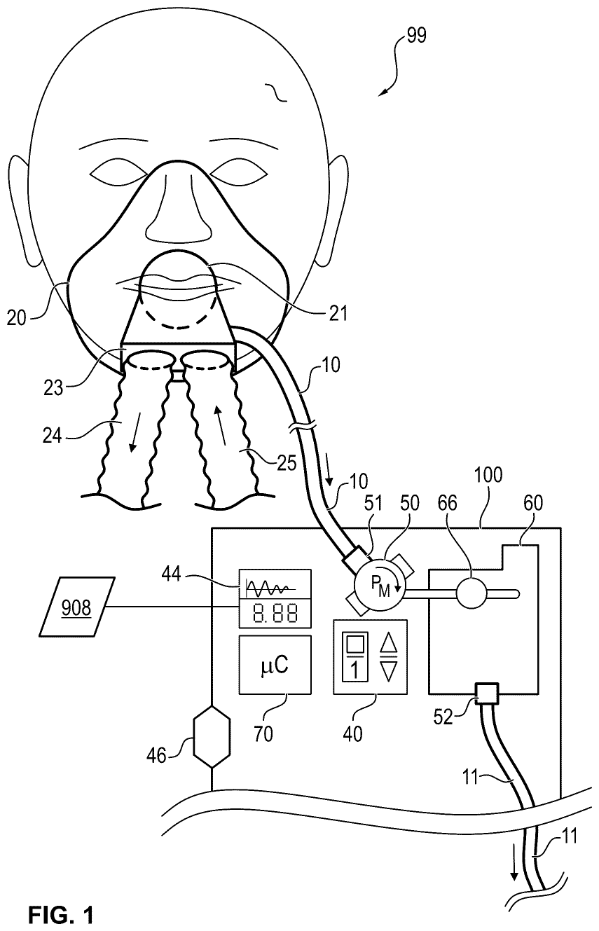

Referring to the drawings, identical elements are designated with identical reference numerals. In the description of FIGS. 1, 2, 3, 4, reference is made alternately to explanations of the overall functionality of the breathing mask, monitoring system and catalytic gas measuring device.

FIG. 1 shows a schematic representation of a monitoring system 100 with some features according to US20210405008 A1 along with operating and input elements 40, display elements 44, control unit 70, interface 46, output signal 908, which enables control and/or configuration of the operation of monitoring system and gas measuring device, as well as connection features including gas outlet 52 and a connecting line 11 to form a part of a monitoring system and gas measuring device system according to the invention. The monitoring system 100 is connected to a person 99 via a sample gas line 10 by means of a breathing mask 20. The person 99 is an aircraft pilot. The breathing mask 20 has a gas connection 21, a connection element 23 and hose lines 24, 25. The hose lines 24, 25 are used to remove and supply breathing gases to the person 99. The monitoring system 100 has operating and input elements 40, display elements 44, at least one module P_{M} for gas delivery 50, a sensor system 60 with at least one sensor 66. The at least one sensor 66 is configured as a paramagnetic gas sensor 60, in particular as a paramagnetic oxygen sensor 60. The module P_{M} for gas delivery 50 is preferably configured as a pump P_{M}. In addition, the monitoring system 100 has a control unit 70.

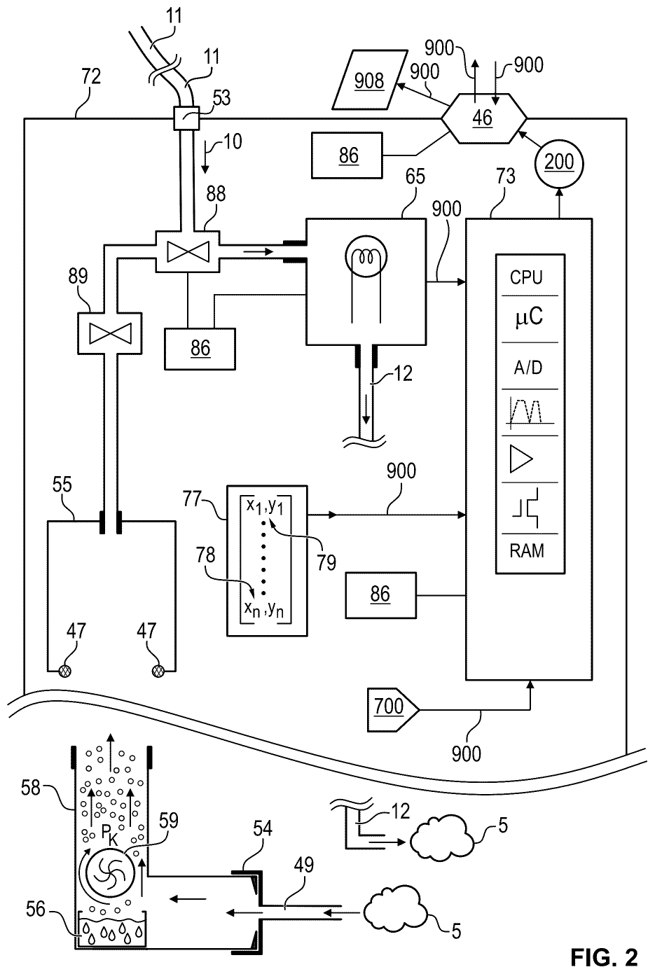

The operating and input elements 40, the display elements 44, the sensors 60 and the module P_{M} for gas delivery 50 are connected to the control unit 70 via signal and data lines or control lines, which are not shown in FIGS. 1 to 2. These control lines or signal and data lines can, for example, be configured as a bus system or network.

The control unit 70 is configured and intended to control and/or actuate the module P_{M} for gas delivery 50 in such a way that breathing gases or breathing gas mixtures 10 are delivered from the breathing mask 20 through the sample gas line 10 and a gas inlet 51 to the sensor system 60.

Thus, a quantity or partial quantity of breathing gas mixture 10 is then available to the at least one sensor 66 in order to detect and/or analyze it metrologically and provide it to the control unit 70 as measured values. The control unit 70 enables the measured values to be evaluated, processed and displayed on display elements 44. A quantity of breathing gas 10 is passed to a gas inlet 53 of a catalytic gas measuring device 72 via a schematically shown gas outlet 52 and a connecting line 11. The operating and input elements 40 make it possible to configure the monitoring system 100 and/or the gas measuring device 72 of the monitoring system 100 and gas measuring device 72.

In addition, a data interface 46 is provided on the gas measuring device, which enables control and/or configuration of the operation 900 of monitoring system 100 and gas measuring device 72.

FIG. 2 shows the monitoring system 100 according to FIG. 1 in combination with a catalytic gas measuring device 72. The catalytic gas measuring device 72 has a catalytic gas sensor 65. Quantities of breathing gas mixture 10 can flow as a gas sample via the connecting line 11 from the monitoring system 100 via a gas inlet 53 to the catalytic gas measuring device 72 and with the aid of switching device and/or changeover device 88, which can be in the form of valves or ⅔-way valves, for example, to the catalytic gas sensor 65.

According to FIG. 2, an evaluation unit 73 is configured as a submodule of the control unit 70 of the monitoring system 100 by means of at least one data interface 46 to carry out coordination 900 and cooperation between the monitoring system 100 and the gas measuring device 72 as well as an exchange of data and/or instructions with the evaluation unit 73 via a data interface 46.

A data memory 77 is assigned to the evaluation unit 73. Conversion factors 79 for one or a plurality of constellations of gases, gas mixtures, fluids as target gases can be stored as data records 78 in the data memory 77.

The catalytic gas measuring device 72 is configured with the catalytic gas sensor 65 and the evaluation unit 73 for a metrological determination of at least one further gas, fluid or gas mixture selected as a target gas in the supplied quantities of breathing gas or breathing gas mixture 10.

The evaluation unit 73 is furthermore configured to perform the metrological determination of a presence of the at least one further gas, fluid or gas mixture selected as target gas in the supplied quantities of breathing gas or breathing gas mixture 10, taking into account the specific conversion factor 79 assigned to the at least one further gas, fluid, gas mixture or group of gases, fluids or gas mixtures selected as target gas and stored as data set 78 in the data memory 77. Finally, the evaluation unit 72 is configured to determine a target gas identifier 200, which indicates the presence of the target gas in the breathing gas mixture 10, and to provide it as an output signal 908 at the output unit 44 and/or the data interface 46.

A calibration interface 55 is arranged on the catalytic gas measuring device 72 as a further gas inlet. Sealing elements 47 are shown, which are intended to schematically illustrate the gas-tight coupling of calibration interface 55 to the evaporation chamber 58. An evaporation chamber 58 and a further module P_{K} 59 for gas delivery are arranged in or on the calibration interface 54 according to this FIG. 2.

The evaporation chamber 58 is configured to accommodate an evaporation tray 56. The evaporation tray 56 is configured and prepared to hold a defined quantity of a calibration gas as a calibration fluid in liquid form at ambient pressure.

The evaporation chamber 58 is provided with a cover element 54; the evaporation chamber 58 can be closed by the cover element 54 after the evaporation tray 56 has been filled with a defined quantity of a specific calibration fluid. Quantities or partial quantities of the calibration fluid evaporate in a defined manner in the evaporation chamber 58—preferably until saturation in the volume of the evaporation chamber 58—and thus form the calibration gas with a defined concentration.

In FIG. 2, an opening as gas access 49 to the environment 5 is shown by way of example as an element of the cover element 54; in optional embodiments, such openings can also be formed as part of the evaporation chamber 58.

The further module P_{K} 59 for gas delivery is configured to supply defined quantities of air from an environment 5 indirectly through the gas access 49, the evaporation chamber 58 and via the evaporation tray 56, the calibration interface 55 and the switching device 88, 89 to the catalytic gas sensor 65. With the aid of switching device 89, which can be in the form of valves, for example, quantities of calibration gas can be released, activated and deactivated for an inflow via the calibration interface 55 to the catalytic gas sensor 65.

The further gas delivery module P_{K} 59 can, for example, be configured as a blower or a pump, which allows defined quantities of air from the environment 5 to flow into the evaporation chamber 58 for a predetermined period of time. In the evaporation chamber 58, this quantity of air is enriched with evaporated calibration fluid from the evaporation tray 56. This results in a quantity of air enriched with the calibration fluid, which is then fed to the catalytic gas sensor 65 as calibration gas by means of the further gas delivery module P_{K} 59.

This provides the catalytic gas sensor 65 with a defined calibration gas of a defined concentration for calibration.

In addition, it is possible for the control unit 70 or the evaluation unit 73 to include special states 700 in the control and coordination of the module P_{M} 50 for gas delivery, the switching or changeover device 88 when supplying quantities of breathing gas mixture 10 to the gas measuring device 72 to the catalytic gas sensor 65.

Such situations include, for example, conditions 700 in which it may be assumed that a gas, fluid or gas mixture other than oxygen, nitrogen, moisture or water vapor and carbon dioxide may be present in the breathing gas mixture 10.

Such special situations 700 can serve the control unit 70 or the evaluation unit 73 as a trigger event in order to activate the switching device or switching device 88, 89 as well as components of a power supply 86 of the gas measuring device 72 in such a way as to enable a supply of quantities of breathing gas mixture 10 to the catalytic gas sensor 65 and to initiate analysis and metrological determination of the target gas. By means of the switching device or changeover device 88, 89, for example, a supply of quantities of calibration gas, a distribution and/or division of quantities of breathing gas mixture 10 as a gas sample between the catalytic gas sensor 65 and a feed-through or discharge by means of a connecting line 12 into the environment 5, for example a cockpit of an aircraft, can take place.

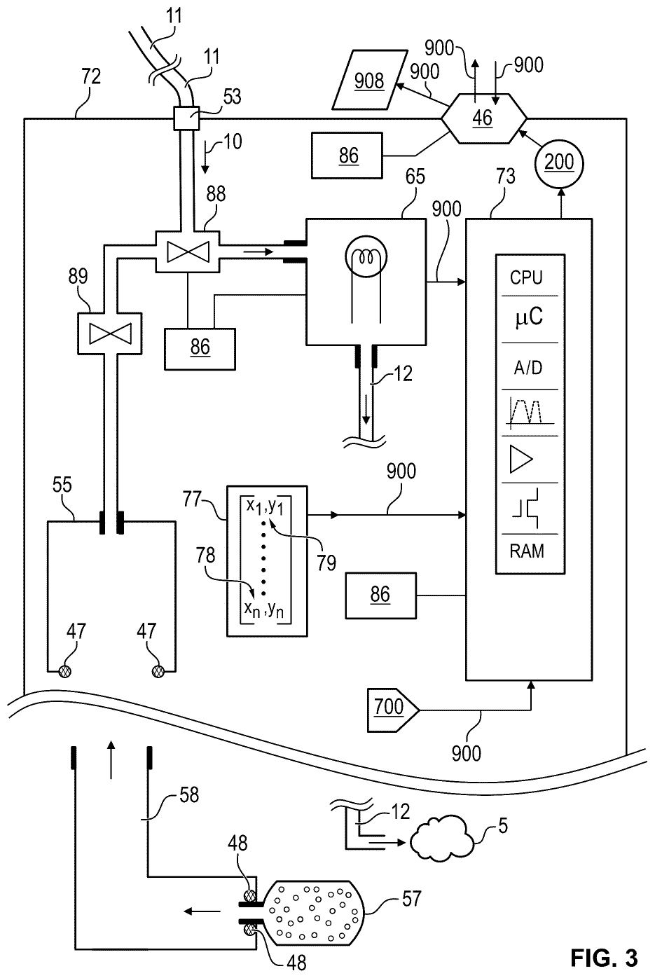

FIG. 3 shows the monitoring system 100 according to FIG. 1 in combination with an alternative catalytic gas measuring device 72. The catalytic gas measuring device 72 has a catalytic gas sensor 65. The function of the catalytic gas measuring device 72 in measuring mode with the associated functions of data memory 77, data records 78, conversion factors 79, evaluation unit 73, data interface 46, connection line 11, gas inlet 53, switching device, switching device 88, output unit 44 is the same as described for FIG. 2.

The same elements in FIGS. 1, 2, 3 are designated with the same reference numbers in FIGS. 1, 2 and FIG. 3.

In contrast to FIG. 2, in FIG. 3 the catalytic gas measuring device 72 is configured with a calibration interface 55 with an alternative configuration of the evaporation chamber 58. Sealing elements 47 are shown, which are intended to schematically illustrate the gas-tight coupling of the calibration interface 55 to the evaporation chamber 58. The evaporation chamber 58 is configured to accommodate a calibration gas cylinder or a calibration gas bottle 57. The calibration gas cylinder 57 can take the form of a cylinder, a cartridge or a cassette cartridge and can be configured to hold a defined volume of a calibration gas as a calibration fluid in gaseous or liquid form at a pressure level above the ambient pressure. Sealing elements 48 are shown, which are intended to schematically illustrate the gas-tight coupling of the calibration gas bottle 57 to the evaporation chamber 58.

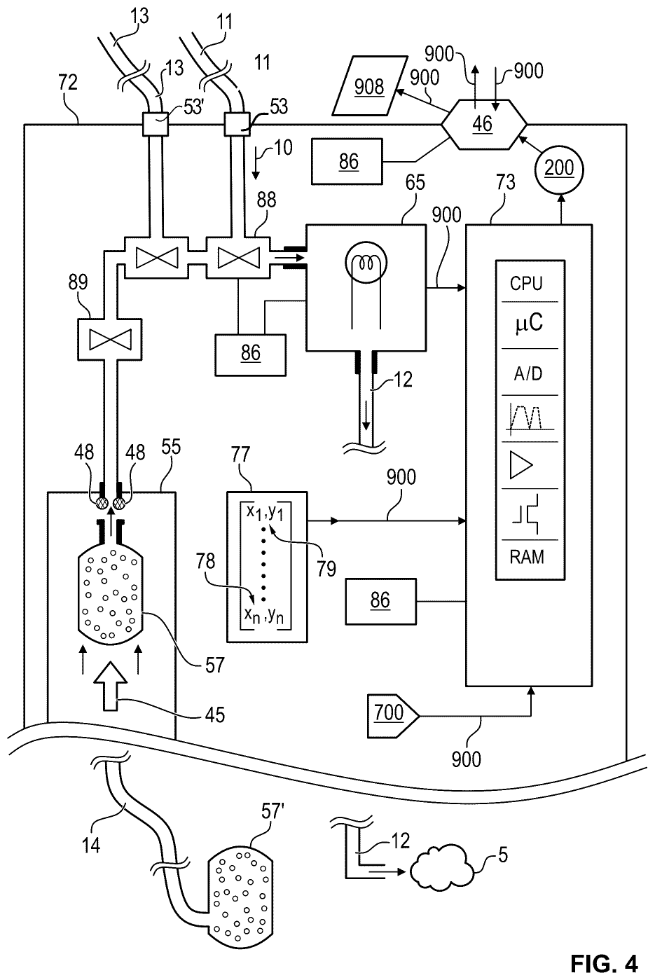

FIG. 4 shows the monitoring system 100 according to FIG. 1 in combination with a further alternative catalytic gas measuring device 72. The catalytic gas measuring device 72 has a catalytic gas sensor 65.

The function of the catalytic gas measuring device 72 in measuring mode with the associated functions of data memory 77, data records 78, conversion factors 79, evaluation unit 73, data interface 46, connecting line 11, gas inlet 53, switching device, switching device 88, output unit 44 is the same as described for FIG. 2. The same elements in FIGS. 1, 2, 3, 4 are designated with the same reference numbers in FIGS. 1, 2, 3 and FIG. 4.

In contrast to FIG. 2 and FIG. 3, FIG. 4 shows a further alternative configuration of the calibration interface 55 on the catalytic gas measuring device 72, which is configured to directly accommodate a calibration gas bottle 57 instead of the evaporation chamber 58 (FIG. 2).

The calibration gas bottle 57 can be configured in the form of a cylinder, a cartridge or a cassette cartridge and can be configured and intended to hold a defined volume of a calibration gas as a calibration fluid in gaseous or liquid form at a pressure level above the ambient pressure. Sealing elements 48 are shown, which are intended to schematically illustrate the gas-tight coupling of the calibration gas bottle 57 to the calibration interface 55. A feed direction 45 is shown schematically to illustrate the establishment of a direct connection between calibration interface 55 and calibration gas bottle 57.

FIG. 4 shows two further optional configuration variants of the catalytic measuring device 72 and the calibration interface 55.

On the one hand, a further sample gas line 13 is shown, via which a further sample gas can also be supplied to the catalytic gas sensor 65 if required. This enables, for example, a parallel, alternating supply of gas samples from the cockpit (to gas inlet 53′ via the gas sample line 13) and the breathing mask (to gas inlet 53 via the gas sample line 10 and the connecting line 11).

Furthermore, a calibration gas line 14 is shown, which enables a spatially remote connection of an alternative calibration gas bottle 57′ to the calibration interface. An application using a spatially remote calibration gas bottle 57′ with an exemplary length of the calibration gas line 14 of 1.5 to 2.5 meters is advantageous compared to a direct connection of the calibration gas bottle 57 in situations in which a calibration fluid must be heated or vaporized before use.

FIGS. 2 to 4 thus show how calibrations can be configured and effected for the catalytic measuring device 72 in a variety of adaptations to the respective boundary conditions (ambient temperature, type of calibration gas, availability of the calibration gas, aggregate state of the calibration gas/fluid) and situations (aircraft type, outdoor use on the airfield, indoor use in the hangar or in the maintenance hangar).

While specific embodiments of the invention have been shown and described in detail to illustrate the application of the principles of the invention, it will be understood that the invention may be embodied otherwise without departing from such principles.

### REFERENCE LIST

5 Surroundings (environment)
10, 13 Sample gas line
14 Calibration gas line
11, 12 Connecting lines
20 Breathing mask
21 Gas connection to breathing mask
24, 25 Hose lines
40 Control elements
44 Display elements
45 Feed direction, feed
46 Data interfaces
47, 48 Sealing elements
50 First module for gas extraction, pump P_{M1}
49, 51, 53 Gas inlet
52 Gas outlet
53, 53′ Gas inlet
54 Cover element, lid, flap
55 Calibration interface, slide-in unit, cassette
56 Evaporation tray (dish)
57, 57′ Calibration gas cylinder, calibration gas bottle
58 Evaporation chamber
59 Additional module for gas extraction, blower, pump P_{K}
60 Sensors
65 Catalytic gas sensor
66 Gas sensor, paramagnetic oxygen sensor
70 Control unit
72 Catalytic gas measuring device
73 Evaluation unit
77 Data memory
78 Data set, data records
79 Conversion factor
86 Components of the electrical power supply
88, 89 Switching device, valves
99 Person, pilot, co-pilot, aircraft pilot
100 Monitoring system
200 Target gas identifier
700 Special condition, situation
900 Operation, control and coordination of operations
908 Output signal

What is claimed is:

1. A system comprising:
a monitoring system configured to monitor a gas composition of breathing gases or breathing gas mixtures in aircraft, the monitoring system comprising: a paramagnetic oxygen sensor; a gas transport module for gas transport; and a control unit;
a catalytic gas measuring device associated with the monitoring system, the catalytic gas measuring device comprising a catalytic gas sensor; an evaluation unit; and a calibration gas interface;
wherein the control unit is configured to organize, monitor, and control or regulate a process of metrological monitoring of the gas composition of air, breathing air, breathing gases or breathing gas mixtures in aircraft or aircraft devices and to coordinate an operation of monitoring systems and catalytic gas measuring device,
wherein the gas transport module for gas transport is configured for gas transport by means of a sample gas line to supply quantities of breathing gas or breathing gas mixture from a measuring location through a gas inlet on the monitoring system to the paramagnetic oxygen sensor and further to the catalytic gas measuring device,
wherein the monitoring system with the paramagnetic oxygen sensor and the control unit is configured to perform a qualitative and a quantitative metrological detection of a concentration of oxygen in the breathing gas or breathing gas mixture,
wherein the catalytic gas measuring device and the evaluation unit are configured for a metrological determination of at least one further selected gas or gas mixture as a selected target gas,
wherein the evaluation unit is configured to carry out the metrological determination of a presence of the target gas in the supplied breathing gas or breathing gas mixture, to determine a target gas identifier and to provide the target gas identifier as an output signal which indicates a presence of the selected target gas in the breathing gas or breathing gas mixture, and
wherein the calibration gas interface is arranged on the catalytic gas measuring device and is configured to provide predetermined quantities of calibration gas to the catalytic gas sensor.

2. A system according to claim 1,
wherein the catalytic gas measuring device further comprises a data memory which is connected to the evaluation unit,
wherein at least one specific conversion factor for the at least one calibration gas associated with the selected target gas is stored in the data memory, and
wherein the evaluation unit is configured to carry out the determination of a proportion of the selected target gas in the breathing gas or breathing gas mixture, based on the specific conversion factor, and to carry out a scaling based on the specific conversion factor corresponding to the selected target gas and to include the scaling based on the specific conversion factor in the determination of the target gas identifier.

3. A system according to claim 1, wherein an evaporation chamber is arranged in or at the calibration interface as calibration medium, the evaporation chamber or the calibration interface being configured to accommodate a calibration gas cylinder, the calibration gas cylinder being configured to store quantities of a calibration gas at a pressure level above ambient pressure.

4. A system according to claim 1,
wherein a receptacle for an evaporation chamber is arranged in or at the calibration interface,
wherein the evaporation chamber is configured to receive an evaporation tray,
wherein the evaporation tray is configured to hold and/or store quantities of a calibration fluid, and
wherein a further gas transport module for gas transport is arranged in or at the calibration interface or in or at the evaporation chamber, which further gas transport module is configured to transport quantities of calibration gas from the evaporation chamber to the catalytic gas sensor.

5. A system according to claim 1, wherein a data interface is provided with the monitoring system and/or with the catalytic gas measuring device, which data interface is configured to indicate and enable a provision of information on a target gas, on a calibration gas and/or on the specific conversion factor to the catalytic gas measuring device.

6. A system according to claim 1, wherein the control unit determines, based on the target gas identifier provided by the catalytic gas measuring device, whether, during operation of the breathing gas supply of the aircraft, there a current situation which is hazardous to the health of an aircraft pilot is present, and, in the event that the current situation which is hazardous to the health of an aircraft pilot is present, activates a vibration alarm detector arranged in or on the monitoring system or a vibration alarm detector assigned to the monitoring system.

7. A system according to claim 1, further comprising an interface arrangement interconnecting components of the monitoring system and the catalytic gas measuring device,
wherein the interface arrangement comprises one or more data interfaces and line connections, and
wherein the interface arrangement and the control unit are configured to carry out a coordination of catalytic gas measuring device and monitoring system.

8. A system according to claim 7, wherein the control unit is configured, when carrying out the coordination of monitoring system and catalytic gas measuring device to execute:
an activation of a switching device or changeover device for starting a supply of quantities of calibration gas and/or breathing gas to the catalytic gas measuring device; or
a deactivation of the switching device or changeover device for terminating the supply of quantities of calibration gas and/or breathing gas to the catalytic gas measuring device, or
an activation of one or more of components of the catalytic gas measuring device, which components of the catalytic gas measuring device comprise the evaluation unit, the catalytic gas sensor and power supply component; or
a deactivation of the components of the catalytic gas measuring device, or
a connection of the electrical power supply of the monitoring system and/or gas measuring device or individual components of the monitoring system and/or the catalytic gas measuring device; or
a switching off of the electrical power supply to the monitoring system and/or gas measuring device or individual components of the monitoring system and/or the catalytic gas measuring device.

9. A system according to claim 1, wherein the monitoring system and/or on the catalytic gas measuring device further comprises an input unit configured to allow an input or a selection of a selected gas or gas mixture as target gas and/or of a calibration gas and/or of the specific conversion factor.

10. A system according to claim 1, wherein the monitoring system and/or on the catalytic gas measuring device further comprises an output unit, wherein the output unit is configured to provide and/or visually output with respect to the selected target gas and/or with respect to the selected calibration gas and/or to output status information with respect to the catalytic gas measuring device.

11. A system according to claim 1, wherein the evaluation unit is configured as a submodule of the control unit of the monitoring system.

12. A system according to claim 1, wherein the catalytic gas measuring device is integrated in the monitoring system as a module of the monitoring system or is arranged as a further, additional module, at the gas outlet of the monitoring system.
